(19) United States
(12) Patent Application Publication
(10) Pub. No.: US 2017/0086677 A1
(43) Pub. Date: Mar. 30, 2017

(54) APPARATUS AND SYSTEM TO IDENTIFY A BLOOD PRESSURE CUFF SIZE

(71) Applicant: GENERAL ELECTRIC COMPANY, Schenectady, NY (US)

(72) Inventors: Otto Valtteri Pekander, Espoo (FI); Kristian Matti Karru, Kirkkonummi (FI)

(21) Appl. No.: 14/870,787

(22) Filed: Sep. 30, 2015

(51) Int. Cl. A61B 5/00; A61B 5/022

(52) U.S. Cl. CPC A61B 5/0082; A61B 5/02233

(57) ABSTRACT

The present application discloses a system for monitoring blood pressure. The system comprises a blood pressure cuff including a connector having an identifier associated with a blood pressure cuff size and a blood pressure measurement device comprising an emitter and a detector, the detector operatively connected to a processor. When the blood pressure cuff connector is mated with the measurement device, the emitter is positioned to direct radiation at the identifier and the detector is positioned to receive radiation from the identifier and generate a signal. The processor then determines the blood pressure cuff size based on the signal received from the detector.

### BACKGROUND OF THE INVENTION

The subject matter disclosed herein relates to non-invasive blood pressure (NIBP) monitoring. More specifically, the present disclosure relates to an apparatus and system to identify a blood pressure cuff size.

Automated blood pressure monitoring has rapidly become an accepted and central aspect of human health care. Such monitors are now a conventional part of patient monitoring, especially in clinics, emergency rooms, intensive and critical care units, and in the operating room. As no single cuff size is effective for all possible patients, cuffs of various sizes ranging from infant or child, to large adult are used. Additionally, blood pressure measurements may be obtained at different locations on the body, such as the thigh, and additional specially sized cuffs are may be used. Using the properly sized blood pressure cuff is important because erroneous blood pressure measurements may result from using a cuff that is too large or too small.

Therefore, the appropriately sized cuff needs to be selected by the clinician, and the cuff size needs to be identified by the blood pressure measurement device. In many blood pressure monitors, the cuff size must be manually set or selected by a clinician. In other devices, the cuff size may be automatically identified via RFID, flow resistors, or by detecting cuff volume. Each of these methods, however, is flawed. Manual identification is ripe for user error. More automatic solutions, such as flow resistors, have a negative impact on performance, price and the manufacturing process by adding complexity to the blood pressure cuff.

Therefore, a reliable system and method for identifying a blood pressure cuff size is desired.

### BRIEF DESCRIPTION OF THE INVENTION

The above-mentioned shortcomings, disadvantages and problems are addressed herein which will be understood by reading and understanding the following specification.

In an embodiment, a blood pressure measurement device for use with a blood pressure cuff having an identifier comprises an electromagnetic radiation emitter positioned to direct radiation onto the identifier; an electromagnetic radiation detector positioned to detect the radiation from the identifier; and a processor operatively connected to the detector, wherein the detector sends a signal to the processor, and the processor determines a cuff size based on the signal.

In another embodiment, a blood pressure cuff comprises a connector having an identifier corresponding to a blood pressure cuff size.

In another embodiment, a system for monitoring blood pressure comprises a blood pressure cuff including a connector having an identifier associated with a blood pressure cuff size and a blood pressure measurement device comprising an emitter and a detector, the detector operatively connected to a processor. When the blood pressure cuff connector is mated with the measurement device, the emitter is positioned to direct radiation at the identifier and the detector is positioned to receive radiation from the identifier and generate a signal. The processor then determines the blood pressure cuff size based on the signal received from the detector.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a non-invasive blood pressure measurement system attached to a patient in accordance with an embodiment of the disclosure;

FIG. 2 is a schematic diagram of a plurality of connectors in accordance with an embodiment of the disclosure;

FIG. 3 is a schematic diagram of a blood pressure measurement device in accordance with an embodiment of the disclosure;

FIG. 4 is a schematic diagram of a blood pressure measurement device in accordance with an embodiment of the disclosure;

FIG. 5 is a schematic diagram of a blood pressure measurement device in accordance with an embodiment of the disclosure;

FIG. 6 is a schematic diagram of a blood pressure measurement device in accordance with an embodiment of the disclosure; and

FIG. 7 is a flow chart illustrating a method of determining a blood pressure cuff size in accordance with an embodiment of the disclosure.

### DETAILED DESCRIPTION OF THE INVENTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives, and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

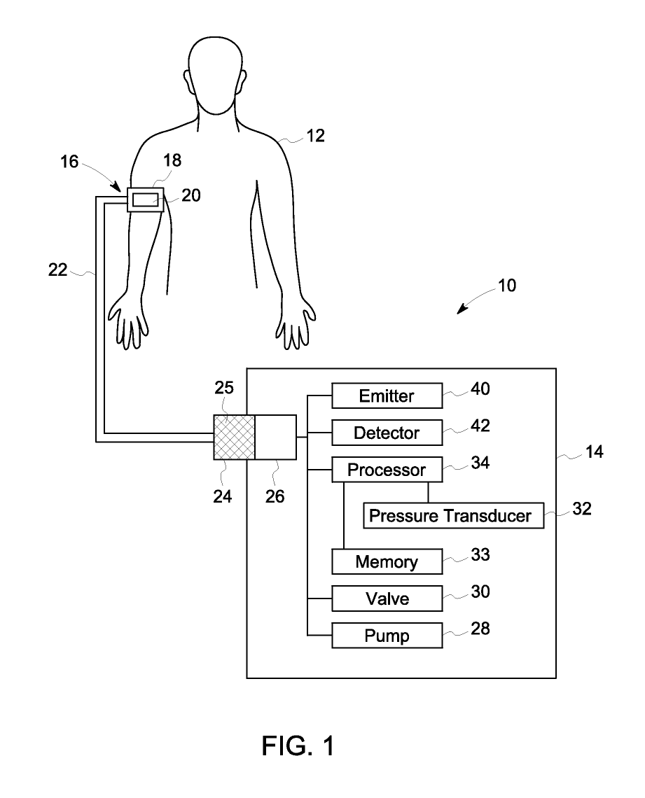

Referring to FIG. 1, a non-invasive blood pressure (NIBP) monitoring system 10 attached to a patient 12 is shown in accordance with an embodiment. The NIBP monitoring system 10 includes a blood pressure measurement device 14 that is releasably connected to a blood pressure cuff 16. The blood pressure cuff 16 includes a flexible band 18. The flexible band 18 is generally wrapped around a limb of patient 12. In FIG. 1, the limb is depicted as comprising the patient's upper arm, however, it should be appreciated that the flexible band 18 may alternatively be applied to other locations (e.g., forearm) and other limbs (e.g., leg).

The flexible band 18 includes an inflatable bladder 20. Although the cuff bladder 20 is shown as being an integral component of the flexible band 18, it should be appreciated that alternative blood pressure cuff configurations may include a separate inflatable cuff bladder.

The blood pressure cuff 16 may be provided in a variety of sizes to accommodate patients of varying sizes as well as different limbs upon which blood pressure monitoring is made. The blood pressure cuff size is based on the circumference of the patient limb around which the blood pressure cuff 16 is placed and corresponds to the length of the flexible band 18. The blood pressure cuff size can range from a neonatal class of cuffs at approximately 3 cm to 8 cm, to an infant class of cuffs at approximately 7 cm to 15 cm, to a child class of cuffs at approximately 12 cm to 21 cm, to an adult class of cuffs at approximately 20 cm to 45 cm, and to a thigh class of cuffs at 40 cm to 60 cm. Additionally, within each class of cuffs, there may be multiple sizes. For example, there may be 3 different neonatal cuffs: a small, medium and large, each sized for a range within the neonatal class.

The blood pressure cuff 16 further comprises a flexible tube 22. Flexible tube 22 is operatively connected at a first end to the cuff bladder 20 and allows air to be pumped into, and released from cuff bladder 20. It should be appreciated that while a single flexible tube 22 is depicted in FIG. 1, the blood pressure cuff 16 may comprise two flexible tubes 22. In such an embodiment, a first flexible tube 22 would direct air into the cuff bladder 20, and while air would be released from the cuff bladder 20 via a second flexible tube 22.

The flexible tube 22 comprises a connector 24 at its second end. The connector 24 is configured to releasably mate with of the blood pressure measurement device 14. In one embodiment, the connector 24 mates in a snap-fit manner with a connector 26 of the blood pressure measurement device 14. In another embodiment, the connector 24 mates in a force-fit manner with connector 26 of the blood pressure measurement device 14. It should be appreciated that the connectors 24 and 26 may releasably mate in other manners, such as a twist-fit manner, a leur locking or a leur slipping manner, within the scope of this disclosure. Connector 24 may be either a male or female connector within the scope of this disclosure and connector 26 would be the corresponding female or male connector.

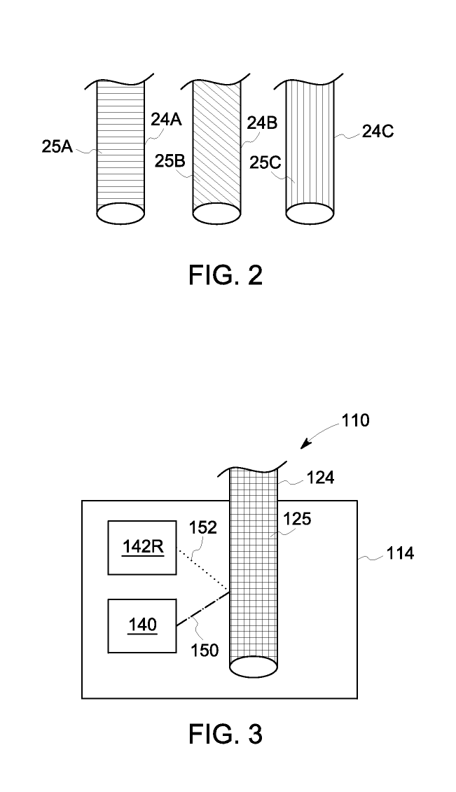

Connector 24 may comprise an identifier 25, represented in FIG. 1 by crosshatching. The identifier 25 may correspond to a blood pressure cuff size. In one embodiment, the identifier is a color in the visible range (approximately 380-800 nm) of the electromagnetic radiation spectrum. For example, connector 25 may be comprised of a colored plastic, nylon or other petroleum-based synthetic material. Referring to FIG. 2, a plurality of connectors 24 are shown in accordance with an embodiment. Connector 24a is a blue connector and the identifier 25a is blue. Connector 24b is a green connector and the identifier 25b is green. Connector 24c is a red connector and the identifier 25c is red. Each of the colors corresponds to a particular cuff size. While blue, green and red are used as examples, it should be appreciated that various other colors in the visible spectrum may be used to identify a specific cuff size.

In another embodiment, the identifier is a coating. For example, a coating within the ultraviolet wavelength range may be applied to the connector 24. In another example, a coating within the infrared wavelength range may be applied to the connector 24. In yet another example, a coating within the visible wavelength range may be applied to the connector 24. As with the colors described above, a plurality of coatings may be used as identifiers, with each coating corresponding to a specific cuff size.

In yet another embodiment, the identifier is a surface texture. For example, the identifier may be a ridged texture with the peaks and valleys spaced at a specific interval. In another example, the identifier may be a honeycomb type texture. In yet another example, the identifier may be a perforated-type texture. Again, as with the colors described above, a plurality of textures may be used as identifiers, with each texture corresponding to a specific cuff size.

It should be appreciated that other types of identifiers or combinations thereof may be used to differentiate between cuff sizes. For example, the identifier may be a color-based pattern.

Turning back to FIG. 1, the blood pressure monitoring device 14 includes a pump 28 adapted to inflate the cuff bladder 20, and one or more valves 30 adapted to deflate the cuff bladder 20. The blood pressure monitoring device 14 includes a pressure transducer 32 operable to sense or identify pressure pulses at the portion of the limb to which the blood pressure cuff 16 is attached. A processor 34 converts the pressure pulse data from the pressure transducer 32 into blood pressure data. The processor 34 may be a microprocessor and other circuitry that retrieves and executes software.

The blood pressure measurement device 14 is configured to measure mean arterial pressure (MAP), systolic blood pressure (SBP), and/or diastolic blood pressure (DBP) by inflating the blood pressure cuff bladder 20 to a supra-systolic pressure level and measuring oscillations under the blood pressure cuff bladder 20 as the cuff bladder 20 is deflated. For purposes of this disclosure, the term “oscillation” refers to a measurable pressure level pulse produced by a change in volume of an artery under the pressure cuff bladder 20.

The blood pressure measurement device 14 further comprises an electromagnetic radiation emitter 40. The emitter 40 is configured to direct radiation onto the connector 24, and specifically the identifier 25, when the connector 24 is mated with the blood pressure measurement device 14. In one embodiment, the emitter 40 may be a light emitting diode (LED) that emits electromagnetic radiation in the visible wavelength spectrum (approximately 380-800 nm). In another embodiment, the emitter 40 may be a LED that emits radiation in the ultraviolet wavelength spectrum (approximately 10-400 nm). In yet another embodiment, the emitter 40 may a LED that emits radiation in the infrared wavelength spectrum (approximately 700 nm-1 mm). It should be appreciated that various other embodiments of the emitter 40 may be envisioned within the scope of this disclosure. For example, the emitter 40 may be a RF source. In another example, the emitter 40 may be configured to emit radiation in a plurality of spectra such as the visible spectrum and the ultraviolet spectrum. In yet another example, the emitter 40 may be an ambient light source.

The blood pressure measuring device 14 also comprises an electromagnetic radiation detector 42. The detector 42 may be a photodetector or any other sensor configured to detect electromagnetic radiation from the emitter 40. Depending on the positioning of the emitter 40 and detector 42 with respect to one another and the mated connector 24 having an identifier 25, the detector 42 may detect radiation by reflection or absorption, or a combination thereof. For example, if the emitter 40 and detector 42 are placed on the same side of the connector 24, the detector 42 may be configured to detect radiation that was reflected off of the identifier 25. If the emitter 40 and detector 42 are placed on opposing sides of the connector 24, the detector 42 may be configured to detect radiation by absorption.

The detector 42 may be configured to detect electromagnetic radiation in a particular spectrum. For example, the detector 42 may detect visible wavelength spectrum (approximately 380-800 nm), the ultraviolet wavelength spectrum (approximately 10-400 nm), or the infrared wavelength spectrum (approximately 700 nm-1 mm). The detector 42 may detect radiation across a plurality of spectra.

The detector 42 is configured to transmit a signal relating to the detected radiation to the processor 34. The processor 34 may be a microprocessors, controller, microcontroller, or other logic based device, or combination thereof, that operate based on instructions stored on a tangible and non-transitory computer readable storage medium, memory 33. The processor 34 is configured to receive the signal from the detector 42 and determine the size of the blood pressure cuff 16 mated with the blood pressure measurement device 14 via computer-readable code stored on a memory 33. The memory 33 may also be configured to store blood pressure measurement data determined by the processor 34.

Referring to FIG. 3, a blood pressure monitoring system 110 according to an embodiment is shown. The blood pressure monitoring system 110 comprises a blood pressure measurement device 114 mated with a connector 124 of a blood pressure cuff (not shown). The blood pressure measurement device 114 comprises an emitter 140 that is configured to direct electromagnetic radiation 150 towards connector 124, and more specifically towards an identifier 125. Connector 124 comprises the identifier 125, depicted in FIG. 3 with cross-hatching, that is associated with a specific blood pressure cuff size. The identifier 125 can be a color, a coating, a texture, or a combination thereof. The blood pressure measurement device 114 also comprises a detector 142R that is positioned to detect electromagnetic radiation 152 that is reflected off of the connector 124. As depicted in FIG. 3, the emitter 140 and the detector 142 are positioned on the same side of connector 124. It should be appreciated, however, that other configurations of the emitter 140 and detector 142 with respect to the connector 124 may be envisioned within the scope of this disclosure. For example, the emitter 140 and detector 142 may be positioned near adjacent sides of the connector 124. The detector 142R is configured to transmit a signal indicative of the reflected electromagnetic radiation 152 to the processor (not shown).

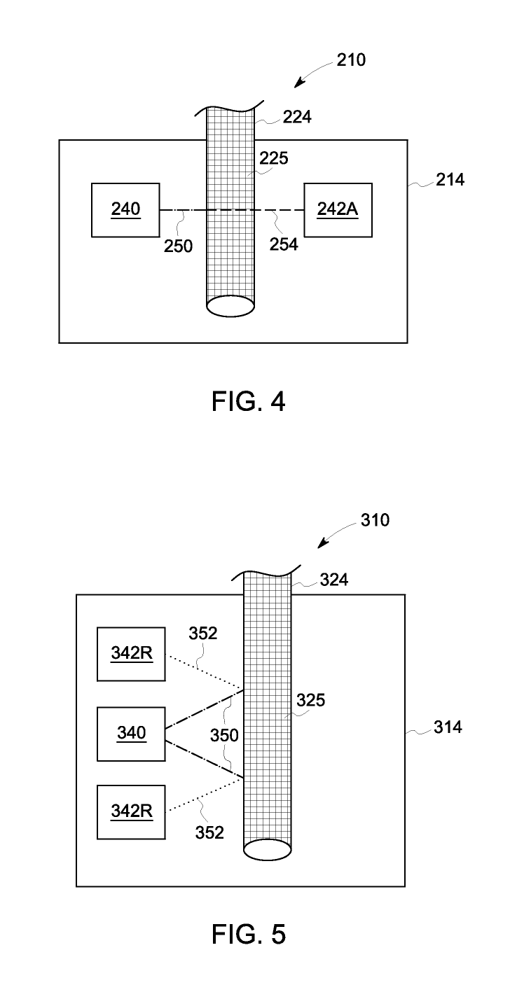

Referring to FIG. 4, a blood pressure monitoring system 210 is shown according to another embodiment. The blood pressure monitoring system 210 comprises a blood pressure measurement device 214 mated with a connector 224 of a blood pressure cuff (not shown). The blood pressure measurement device 214 comprises an emitter 240 that is configured to direct electromagnetic radiation 250 towards connector 224, and more specifically towards an identifier 225. Connector 224 comprises the identifier 225, depicted in FIG. 4 with cross-hatching, that is associated with a specific blood pressure cuff size. The identifier 225 can be a color, a coating, a texture, or a combination thereof. A detector 242A is positioned to detect electromagnetic radiation 254 that is not absorbed by the connector 224. In the depicted embodiment, the emitter 240 and detector 242 are on opposing sides of the connector 224. It should be appreciated, however, that other configurations of the emitter 240 and detector 242 are envisioned within the scope of the disclosure. The detector 242A is configured to transmit a signal indicative of the non-absorbed radiation 254 to the processor (not shown).

Referring to FIG. 5, a blood pressure monitoring system 310 in accordance with another embodiment is shown. The blood pressure monitoring system 310 comprises a blood pressure measurement device 314 mated with a connector 324 of a blood pressure cuff (not shown). The blood pressure measurement device 314 comprises an emitter 340 that is configured to direct electromagnetic radiation 350 towards connector 324, and more specifically towards an identifier 325. Connector 324 comprises the identifier 325, depicted in FIG. 5 with cross-hatching, that is associated with a specific blood pressure cuff size. The identifier 325 can be a color, a coating, a texture, or a combination thereof. In the depicted embodiment, the emitter 340 is configured to emit electromagnetic radiation 350 towards two different positions on connector 324. In one embodiment, the emitter 340 may be emitting radiation in a single spectrum, such as the visible spectrum. In another embodiment, the emitter 340 may be emitting radiation in a plurality of spectra, such as the visible spectrum and the infrared spectrum.

The blood pressure measurement device 314 also comprises a plurality of detectors 342R that are positioned to detect electromagnetic radiation 352 that is reflected off of the connector 324. The depicted embodiment comprises two detectors 342R, however it should be appreciated that additional detectors may be used within the scope of the disclosure. In one embodiment, the detectors 342R may be configured to both detect radiation in the same spectrum, such as the visible spectrum. In another embodiment, the detectors 342R are each configured to detect radiation in separate spectra, such as the visible spectrum and the infrared spectrum. The detectors 342R are both configured to transmit a signal indicative of the detected reflected electromagnetic radiation 352 to the processor (not shown).

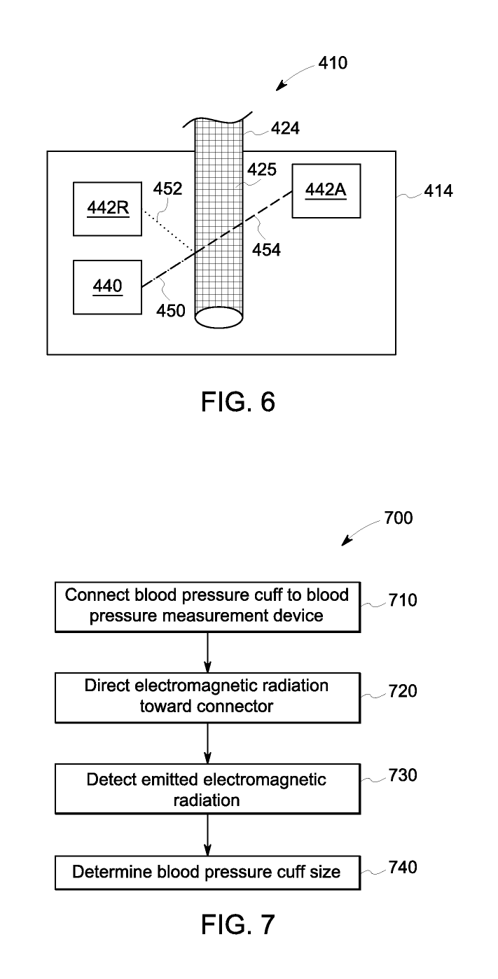

Referring to FIG. 6, a blood pressure monitoring system 410 in accordance with yet another embodiment is shown. The blood pressure monitoring system 410 comprises a blood pressure measurement device 414 mated with a connector 424 of a blood pressure cuff (not shown). The blood pressure measurement device 414 comprises an emitter 440 that is configured to direct electromagnetic radiation 450 towards connector 424, and more specifically towards an identifier 425. Connector 424 comprises the identifier 425, depicted in FIG. 6 with cross-hatching, that is associated with a specific blood pressure cuff size. The identifier 425 can be a color, a coating, a texture, or a combination thereof. The blood pressure measurement device 414 also comprises detectors 442R, 442A. Detector 442R is positioned to detect electromagnetic radiation 452 that is reflected off of the connector 424. Detector 442A is positioned to detect electromagnetic radiation 454 that is not absorbed by the connector 424. Detectors 442R and 442A are configured to send a signal to the processor (not shown) relating to the detected radiation.

Referring to FIG. 7, a method 700 of identifying a blood pressure cuff size is disclosed. For the purposes herein, reference numerals will refer to the system depicted in FIG. 1. It should be appreciated, however that the method may be carried out with any of the systems depicted in FIGS. 3-6.

The method 700 includes a step 710 comprising connecting the blood pressure cuff 16 to the blood pressure measurement device 14. The blood pressure cuff 16 comprises connector 24 having an identifier 25. The identifier 25 is associated with the size of the blood pressure cuff, and more specifically the identifier 25 may be associated with the size of the flexible band 18. The identifier 25 may be a color, a coating, or a texture, or a combination thereof.

The method 700 includes a step 720 comprising emitting with emitter 40 electromagnetic radiation directed towards the connector 24 having an identifier 25. The electromagnetic radiation may be in the visible wavelength spectrum (approximately 380-800 nm), the ultraviolet wavelength spectrum (approximately 10-400 nm), or the infrared wavelength spectrum (approximately 700 nm-1 mm), or any other spectrum of electromagnetic radiation. The emitter may direct electromagnetic radiation of one spectrum or a plurality of spectra towards the connector 24.

The method 700 includes a step 730 comprising detecting with the detector 42 the electromagnetic radiation emitted from the emitter 40. Depending on the positioning of the detector 42 with respect to the emitter 40 and the identifier 25, the detector 42 may detect radiation that is reflected off the connector 24, radiation that passes through the connector 24, or a combination thereof.

The method 700 includes a step 740 comprising determining with the processor 34 the blood pressure cuff size. The processor 34 is configured to receive a signal from the detector 42 indicative of the detected radiation. The processor 34 is configured to associate the detected radiation with a blood pressure cuff size using computer executable code stored on the memory 33.

The apparatus, system and method disclosed herein have several benefits over existing means for identifying blood pressure cuff size. First, it eliminates the user error that is possible in systems that require a manual identification and selection or input of blood pressure cuff size. Second, it produces an economic benefit. No changes or complexities are added to current blood pressure cuff manufacturing processes. Third, system flexibility is increased as a large number of cuff identifiers may be recognized.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A blood pressure measurement device for use with a blood pressure cuff having an identifier, the blood pressure measurement device comprising:
an electromagnetic radiation emitter positioned to direct radiation onto the identifier;
an electromagnetic radiation detector positioned to detect the radiation from the identifier; and
a processor operatively connected to the detector, wherein the detector sends a signal to the processor, and the processor determines a cuff size based on the signal.

2. The device of claim 1, wherein the emitter directs radiation in the visible wavelength range.

3. The device of claim 1, wherein the emitter directs radiation in the ultraviolet wavelength range.

4. The device of claim 1, wherein the detector is positioned to detect radiation by reflection.

5. The device of claim 1, wherein the detector is positioned to detect radiation by absorption.

6. A blood pressure cuff, comprising:
a connector having an identifier corresponding to a blood pressure cuff size.

7. The blood pressure cuff of claim 7, wherein the identifier is a color.

8. The blood pressure cuff of claim 7, wherein the identifier is a coating.

9. The blood pressure cuff of claim 7, wherein the identifier is a texture.

10. A system for monitoring blood pressure, comprising:
a blood pressure cuff comprising a connector having an identifier associated with a blood pressure cuff size; and
a blood pressure measurement device comprising an emitter and a detector, the detector operatively connected to a processor;
wherein when the blood pressure cuff connector is mated with the measurement device, the emitter is positioned to direct radiation at the identifier and the detector is positioned to receive radiation from the identifier and generate a signal, wherein the processor determines the blood pressure cuff size based on the signal received from the detector.

11. The system of claim 10, wherein the emitter directs electromagnetic radiation in the visible wavelength range.

12. The system of claim 10, wherein the emitter directs electromagnetic radiation in the ultraviolet wavelength range.

13. The system of claim 10, wherein the detector is positioned to detect radiation by reflection.

14. The system of claim 10, wherein the detector is positioned to detect radiation by absorption.

15. The system of claim 10, wherein the blood pressure measurement device comprises a first detector positioned to detect radiation by reflection and a second detector positioned to detect radiation by absorption.

16. The system of claim 10, wherein the identifier is a color.

17. The system of claim 10, wherein the identifier is a coating.

18. The system of claim 10, wherein the identifier is a texture.
